(19) United States
(12) Patent Application Publication
(10) Pub. No.: US 2021/0405063 A1
(43) Pub. Date: Dec. 30, 2021

(54) ASSAY DETECTION METHODS FOR VCAM-1 AND CALPROTECTIN

(71) Applicant: PROCISEDX INC., San Diego, CA (US)

(72) Inventors: LIMIN LIU, SAN DIEGO, CA (US); HONGYU CHEN, SAN DIEGO, CA (US); STEFAN WESTIN, SAN DIEGO, CA (US); JINYAO ZHOU, SAN DIEGO, CA (US); MICHAEL HALE, SAN DIEGO, CA (US); LARRY MIMMS, SAN DIEGO, CA (US)

(73) Assignee: PROCISEDX INC., San Diego, CA (US)

(21) Appl. No.: 17/466,939

(22) Filed: Sep. 3, 2021

Related U.S. Application Data
(63) Continuation of application No. PCT/US2020/032919, filed on May 14, 2020.
(60) Provisional application No. 62/848,723, filed on May 16, 2019.
(60) Provisional application No. 62/851,981, filed on May 23, 2019.

(51) Int. Cl. G01N 33/68; G01N 21/64; G01N 33/58

(52) U.S. Cl. CPC G01N 33/6878; G01N 21/6428; G01N 2800/065; G01N 2021/6439; G01N 33/582

(57) ABSTRACT

Assay methods for detecting the presence or amount of VCAM-1 or calprotectin in a sample using fluorescence resonance energy transfer (FRET).

### CROSS-REFERENCES TO RELATED APPLICATIONS

The current application is a continuation of PCT/US2020/032919, filed May 14, 2020, which application claims priority to US Provisional Application Nos. 62/848,723, filed May 16, 2019, and 62/851,981, filed May 23, 2019, the disclosures all of which are hereby incorporated by reference in their entireties for all purposes.

### SEQUENCE LISTING

The instant application contains a Sequence Listing which has been submitted electronically in ASCII format and is hereby incorporated by reference in its entirety. Said ASCII copy, created on Aug. 6, 2020, is named 105741-1189936-029110PC_SL.txt and is 8,578 bytes in size.

### BACKGROUND

Some biological assays rely on time-resolved fluorescence resonance energy transfer (TR-FRET) mechanisms where two fluorophores are used. Biological materials are typically prone to autofluorescence, which can be minimized by utilizing time-resolved FRET. TR-FRET takes advantage of rare earth elements such as lanthanides (e.g., europium and terbium), which have exceptionally long fluorescence emission half-lives. In these assays, energy is transferred between a donor fluorophore and an acceptor fluorophore, if the two fluorophore are in close proximity to one another. Excitation of the donor (e.g., cryptate) by an energy source (e.g. UV light) produces an energy transfer to the acceptor, if the two fluorophores are within a given proximity. In turn, the acceptor emits light at its characteristic wavelength. In order for TR-FRET to occur, the fluorescence emission spectrum of the donor molecule must overlap with the absorption or excitation spectrum of the acceptor chromophore. Moreover, the fluorescence lifetime of the donor molecule must be of sufficient duration to allow TR-FRET to occur.

Cryptates can be used in various bioassays formats. Cryptates are complexes that include a macrocycle within which a lanthanide ion such as terbium or europium is tightly embedded or chelated. This cage like structure is useful for collecting irradiated energy and transferring the collected energy to the lanthanide ion. The lanthanide ion can release the energy with a characteristic fluorescence.

U.S. Pat. No. 6,406,297 is titled “Salicylamide-lanthanide complexes for use as luminescent markers.” This patent is directed to luminescent lanthanide metal chelates comprising a metal ion of the lanthanide series and a complexing agent comprising a salicylamidyl moiety. This patent is hereby incorporated by reference.

U.S. Pat. No. 6,515,113 is titled “Phthalamide lanthanide complexes for use as luminescent markers.” This patent is directed to luminescent lanthanide metal chelates comprising a metal ion of the lanthanide series and a complexing agent comprising a phthalamidyl moiety. This patent is hereby incorporated by reference.

WO2015157057 is titled “Macrocycles” and relates to chemical compounds and complexes that can be used in therapeutic and diagnostic applications. This publication contains cryptate molecules useful for labeling biomolecules. This publication is hereby incorporated by reference.

WO2018130988 discloses cryptates derivatives and conjugates thereof with excellent fluorescent properties. The cryptates are useful in biological assays and methods for the detection and identification of various analytes.

Vascular cell adhesion protein 1 also known as vascular cell adhesion molecule 1 (VCAM-1) or cluster of differentiation 106 (CD106) is a protein that in humans is encoded by the VCAM1 gene. VCAM-1 functions as a cell adhesion molecule. The presence and concentration levels of VCAM-1 are typically measured by an enzyme-linked immunosorbent assay (ELISA).

VCAM-1 solid-phase sandwich ELISA is designed to measure the presence or amount of the analyte bound between an antibody pair. In the sandwich ELISA, a sample is added to an immobilized capture antibody. After a second (detector) antibody is added, a substrate solution is used that reacts with an enzyme-antibody-target complex to produce a measurable signal. The intensity of this signal is proportional to the concentration of target present in the test sample.

Calprotectin is a calcium- and zinc-binding protein and is considered to be neutrophil specific. When there is inflammation in the gastrointestinal (GI) tract and gut, neutrophils release calprotectin, which results in an increased level of calprotectin in the stool. Increased levels of calprotectin can thus be used as a useful diagnostic indicator, with increased levels of calprotectin directly relating to the severity of the inflammation.

Fecal calprotectin is useful in differentiating between IBD (Inflammatory Bowel Disease) and IBS (Irritable Bowel Syndrome). Typically, IBD (e.g. Crohn's Disease (CD) or Ulcerative Colitis (UC)) has accompanying inflammation whereas IBS does not have inflammation. When lower abdominal or gastrointestinal (GI) symptoms are present, a fecal calprotectin test is used to identify whether there is an inflammatory bowel condition and its cause. A higher than normal level of calprotectin indicates inflammation.

In view of the foregoing, what is needed in the art is a homogeneous VCAM-1 assay to provide an increase in flexibility, reliability and sensitivity in addition to higher throughput. In addition, there is a need in the art for new ways to measure and determine the amount of calprotectin in a biological sample The present disclosure provides these and other needs.

### BRIEF SUMMARY

In one embodiment, the present disclosure provides an assay method for detecting the presence or amount of VCAM-1 in a sample, the method comprising:
contacting the sample with a first anti-VCAM-1 antibody having a first epitope to VCAM-1, wherein the first anti-VCAM-1 antibody is labeled with a donor fluorophore;
contacting the sample with a second anti-VCAM-1 antibody having a second epitope to VCAM-1, wherein the second anti-VCAM-1 antibody is labeled with an acceptor fluorophore;
incubating the sample for a time sufficient wherein the first antibody and the second antibody bind VCAM-1 to obtain dual labeled VCAM-1; and
exciting the sample having dual labeled VCAM-1 using a light source to detect a fluorescence emission signal associated with fluorescence resonance energy transfer (FRET).

In another embodiment, the present disclosure provides a method for detecting or quantifying the concentration or level of calprotectin in a sample, comprising:
contacting the sample with a first antibody having a first binding epitope specific to calprotectin, wherein the first antibody is labeled with a donor fluorophore;
contacting the sample with a second antibody having a second binding epitope specific to calprotectin, wherein the second antibody is labeled with an acceptor fluorophore;
incubating the sample for a time sufficient to obtain a dual labeled calprotectin; and
exciting the sample having the dual labeled calprotectin using a light source to detect a fluorescent emission signal associated with fluorescence resonance energy transfer (FRET).

In another embodiment, the present disclosure provides a method for determining inflammation of the gut, comprising:
contacting the sample with a first antibody having a first binding epitope specific to calprotectin, wherein the first antibody is labeled with a donor fluorophore;
contacting the sample with a second antibody having a second binding epitope specific to calprotectin, wherein the second antibody is labeled with an acceptor fluorophore;
incubating the sample for a time sufficient to obtain a dual labeled calprotectin; and
exciting the sample having the dual labeled calprotectin using a light source to detect a fluorescent emission signal associated with fluorescence resonance energy transfer (FRET).

These and other aspects, objects and embodiments will become more apparent when read with the detailed description and figures that follow.

### BRIEF DESCRIPTION OF THE DRAWINGS

FIG. 1 illustrates one embodiment of the present disclosure with a dual labeled analyte.

FIG. 2 illustrates a standard curve using methods of the present disclosure.

FIGS. 3A-B illustrate standard curves using methods of the present disclosure.

FIGS. 4A-B illustrate one embodiment of the present disclosure.

FIG. 5 illustrates a standard curve generated using methods of the present disclosure.

FIG. 6 illustrates a standard curve generated using methods of the present disclosure.

FIG. 7 illustrates a comparison of the methods of the present disclosure with a commercially available method.

FIG. 8 illustrates one embodiment of a donor of the present disclosure.

FIG. 9 illustrates donor and acceptor wavelengths in one embodiment of the present disclosure.

FIG. 10 illustrates one embodiment of an acceptor of the present disclosure.

### DETAILED DESCRIPTION

### I. DEFINITIONS

The terms “a,” “an,” or “the” as used herein not only includes aspects with one member, but also includes aspects with more than one member.

The term “about” as used herein to modify a numerical value indicates a defined range around that value. If “X” were the value, “about X” would indicate a value from 0.9X to 1.1X, and more preferably, a value from 0.95X to 1.05X. Any reference to “about X” specifically indicates at least the values X, 0.95X, 0.96X, 0.97X, 0.98X, 0.99X, 1.01X, 1.02X, 1.03X, 1.04X, and 1.05X. Thus, “about X” is intended to teach and provide written description support for a claim limitation of, e.g., “0.98X.”

When the modifier “about” is applied to describe the beginning of a numerical range, it applies to both ends of the range. Thus, “from about 500 to 850 nm” is equivalent to “from about 500 nm to about 850 nm.” When “about” is applied to describe the first value of a set of values, it applies to all values in that set. Thus, “about 580, 700, or 850 nm” is equivalent to “about 580 nm, about 700 nm, or about 850 nm.”

“Activated acyl” as used herein includes a —C(O)-LG group. “Leaving group” or “LG” is a group that is susceptible to displacement by a nucleophilic acyl substitution (i.e., a nucleophilic addition to the carbonyl of —C(O)-LG, followed by elimination of the leaving group). Representative leaving groups include halo, cyano, azido, carboxylic acid derivatives such as t-butylcarboxy, and carbonate derivatives such as i-BuOC(O)O—. An activated acyl group may also be an activated ester as defined herein or a carboxylic acid activated by a carbodiimide to form an anhydride (preferentially cyclic) or mixed anhydride —OC(O)R^{a} or —OC(NR^{a})NHR^{b} (preferably cyclic), wherein R^{a} and R^{b} are members independently selected from the group consisting of C₁-C₆ alkyl, C₁-C₆ perfluoroalkyl, C₁-C₆ alkoxy, cyclohexyl, 3-dimethylaminopropyl, or N-morpholinoethyl. Preferred activated acyl groups include activated esters.

“Activated ester” as used herein includes a derivative of a carboxyl group that is more susceptible to displacement by nucleophilic addition and elimination than an ethyl ester group (e.g., an NHS ester, a sulfo-NHS ester, a PAM ester, or a halophenyl ester). Representative carbonyl substituents of activated esters include succinimidyloxy (—OC₄H₄NO₂), sulfosuccinimidyloxy (—OC₄H₃NO₂SO₃H), -1-oxybenzotriazolyl (—OC₆H₄N₃); 4-sulfo-2,3,5,6-tetrafluorophenyl; or an aryloxy group that is optionally substituted one or more times by electron-withdrawing substituents such as nitro, fluoro, chloro, cyano, trifluoromethyl, or combinations thereof (e.g., pentafluorophenyloxy, or 2,3,5,6-tetrafluorophenyloxy). Preferred activated esters include succinimidyloxy, sulfosuccinimidyloxy, and 2,3,5,6-tetrafluorophenyloxy esters.

“FRET partners” refers to a pair of fluorophores consisting of a donor fluorescent compound such as cryptate and an acceptor compound such as Alexa 647, when they are in proximity to one another and when they are excited at the excitation wavelength of the donor fluorescent compound, these compounds emit a FRET signal. It is known that, in order for two fluorescent compounds to be FRET partners, the emission spectrum of the donor fluorescent compound must partially overlap the excitation spectrum of the acceptor compound. The preferred FRET-partner pairs are those for which the value RO (Förster distance, distance at which energy transfer is 50% efficient) is greater than or equal to 30 Å.

“Fluorescence resonance energy transfer (FRET)” or “Förster resonance energy transfer (FRET)” refer to a mechanism describing energy transfer between a donor compound such as cryptate and an acceptor compound such as Alexa 647, when they are in proximity to one another and when they are excited at the excitation wavelength of the donor fluorescent compound. A donor compound, initially in its electronic excited state, may transfer energy to an acceptor fluorophore through nonradiative dipole-dipole coupling. The efficiency of this energy transfer is inversely proportional to the sixth power of the distance between donor and acceptor, making FRET extremely sensitive to small changes in distance.

“FRET signal” refers to any measurable signal representative of FRET between a donor fluorescent compound and an acceptor compound. A FRET signal can therefore be a variation in the intensity or in the lifetime of luminescence of the donor fluorescent compound or of the acceptor compound when the latter is fluorescent.

VCAM-1 (vascular cell adhesion protein 1, CD106, INCAM-110) refers to a cell surface sialoglycoprotein expressed by cytokine activated endothelium. VCAM-1 has a number of functions including the regulation of leukocyte migration, leukocyte-endothelial cell adhesion and signal transduction and may play a role in a number of inflammatory diseases. VCAM-1 is distributed across non-leukocyte and leukocyte cells. VCAM-1 is a member of the Ig superfamily of adhesion molecules, is expressed at high levels on cytokine stimulated vascular endothelial cells, and at minimal levels on unstimulated endothelial cells. VCAM-1 is also present on follicular and inter-follicular dendritic cells of lymph nodes, myoblasts, and some macrophages. VCAM1_HUMAN, accession P19320 is SEQ ID NO: 1. VCAM-1 has 739 amino acids and a mass of 81,276 Da. This isoform has been chosen as the ‘canonical’ sequence.

Calprotectin is a calcium- and zinc-binding protein and is considered to be neutrophil specific. When there is inflammation in the gastrointestinal (GI) tract and gut, neutrophils release calprotectin, which results in an increased level of calprotectin in the stool.

### II. EMBODIMENTS

The present disclosure provides a homogenous solution phase time-resolved FRET assay (TR-FRET) to detect VCAM-1 presence or levels in a biological sample such as whole blood. In conjunction with other markers levels, VCAM-1 can be used as an aid in determination of fibrosis in liver diseases such as NASH, Hepatitis C and Hepatitis B. Förster resonance energy transfer or fluorescence resonance energy transfer (FRET) is a process in which a donor molecule in an excited state transfers its excitation energy through dipole-dipole coupling to an acceptor fluorophore, when the two molecules are brought into close proximity, typically less than 10 nm such as, <9 nm, <8 nm, <7 nm, <6 nm, <5 nm, <4 nm, <3 nm, <2 nm, or less than <1 nm. Upon excitation at a characteristic wavelength, the energy absorbed by the donor is transferred to the acceptor, which in turn emits the energy (e.g., in the form of light). The level of light emitted from the acceptor fluorophore is proportional to the degree of donor acceptor complex formation.

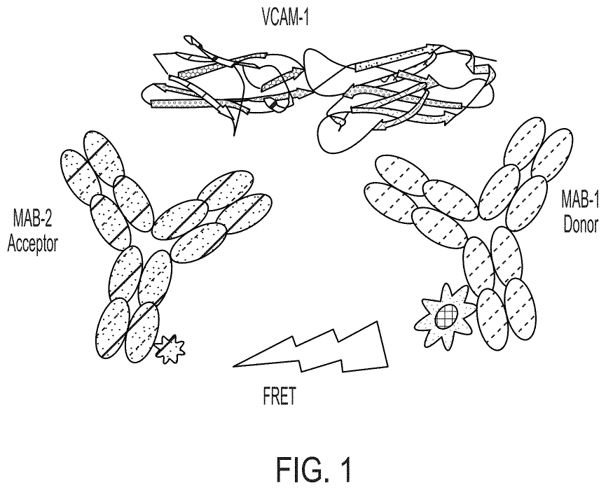

Biological materials are typically prone to autofluorescence, which can be minimized by utilizing time-resolved fluorometry (TRF). TRF takes advantage of unique rare earth elements such as lanthanides, (e.g., europium and terbium), which have exceptionally long fluorescence emission half-lives. Time-resolved FRET (TR-FRET) unites the properties of TRF and FRET, which is especially advantageous when analyzing biological samples. If one anti-VCAM-1 antibody is labeled with a donor fluorophore and a second anti-VCAM-1 antibody is labeled with an acceptor fluorophore, TR-FRET can occur in the presence of the VCAM-1 antigen (FIG. 1).

The use of the FRET phenomenon for studying biological processes implies that each member of the pair of FRET partners will be conjugated to compounds that will interact with one another, and thus bring the FRET partners into close proximity with one another. Upon exposure to light, the FRET partners will generate a FRET signal. In the methods according to the disclosure, the energy donor and the energy acceptor are each conjugated to a different antibody AB-1 and AB-2). The energy transfer between the two FRET partners depends upon each binding to the analyte to generate a dual labeled analyte (a dual labeled VCAM-1). Förster or fluorescence resonance energy transfer (FRET), is a physical phenomenon in which a donor fluorophore in its excited state non-radiatively transfers its excitation energy to a neighboring acceptor fluorophore, thereby causing the acceptor to emit its characteristic fluorescence.

As such, in one embodiment, the present disclosure provides an assay method for detecting the presence and/or amount of VCAM-1 in a sample, the method comprising:
contacting the sample with a first anti-VCAM-1 antibody having a first binding epitope to VCAM-1, wherein the first anti-VCAM-1 antibody is labeled with a donor fluorophore;
contacting the sample with a second anti-VCAM-1 antibody having a second binding epitope to VCAM-1, wherein the second anti-VCAM-1 antibody is labeled with an acceptor fluorophore;
incubating the sample for a time sufficient to obtain dual labeled VCAM-1; and
exciting the sample having dual labeled VCAM-1 using a light source to detect a fluorescence emission signal associated with fluorescence resonance energy transfer (FRET).

Calprotectin is a major protein in neutrophilic granulocytes and macrophages and accounts for much of the protein in the cytosol fraction of these cells. The amount of calprotectin in a sample reflects the amount and number of participating neutrophils in the underlying inflammation. The present disclosure allows for rapid determination of the amount and level of calprotectin and thus the amount of inflammation in the gut.

In one embodiment, the present disclosure provides a method for detecting or quantifying the concentration or level of calprotectin in a sample, comprising:
contacting the sample with a first antibody having a first binding epitope specific to calprotectin, wherein the first antibody is labeled with a donor fluorophore;
contacting the sample with a second antibody having a second binding epitope specific to calprotectin, wherein the second antibody is labeled with an acceptor fluorophore;
incubating the sample for a time sufficient to obtain a dual labeled calprotectin; and
exciting the sample having the dual labeled calprotectin using a light source to detect a fluorescent emission signal associated with fluorescence resonance energy transfer (FRET).

In certain aspects, the sample is obtained from an individual such as a human.

In certain aspects, the sample is a biological sample. Suitable biological samples include, but are not limited to, whole blood, urine, a fecal specimen, plasma or serum. In a preferred aspect, the biological sample is whole blood for VCAM-1 determination. In one aspect, the biological sample is a fecal sample for calprotectin determination.

In certain aspects, the FRET assay is a time-resolved FRET assay. The fluorescence emission signal or measured FRET signal is directly correlated with the biological phenomenon studied. In fact, the level of energy transfer between the donor fluorescent compound and the acceptor fluorescent compound is proportional to the reciprocal of the distance between these compounds to the 6^{th} power. For the donor/acceptor pairs commonly used by those skilled in the art, the distance Ro (corresponding to a transfer efficiency of 50%) is in the order of 1, 5, 10, 20 or 30 nanometers.

In certain aspects, the FRET energy donor compound is a cryptate, such as a lanthanide cryptate.

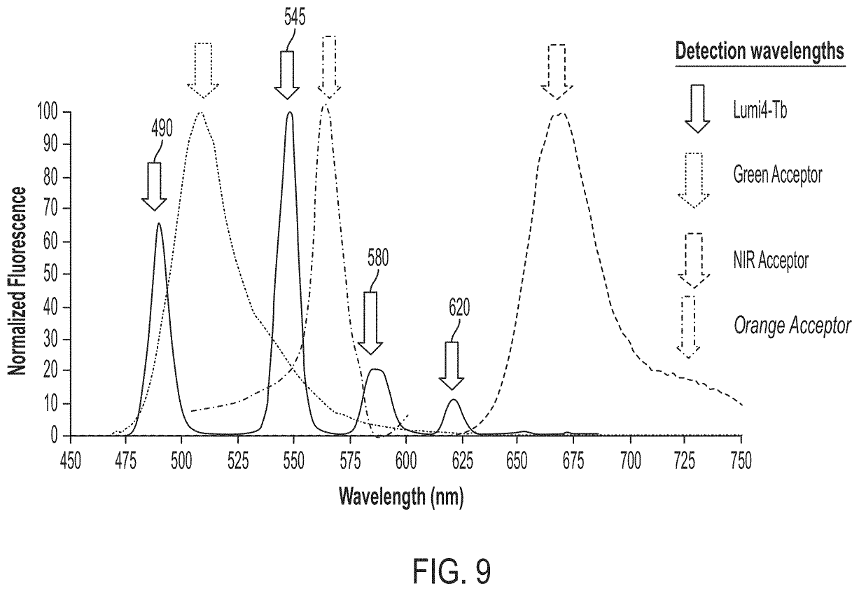

In certain aspects, the cryptate has an absorption wavelength between about 300 nm to about 400 nm such as about 325 nm to about 375 nm. In certain aspects, as shown in FIG. 9, cyptate dyes have four fluorescence emission peaks at about 490 nm, about 548 nm, about 587 nm, and 621 nm. Thus, as a donor, the cryptate is compatible with fluorescein-like (green zone) molecule, Cy5 or DY-647-like (red zone) acceptors to perform TR-FRET experiments.

In certain aspects, the introduction of a time delay between a flash excitation and the measurement of the fluorescence at the acceptor emission wavelength allows to discriminate long lived from short-lived fluorescence and to increase signal-to-noise ratio.

### 1. Vascular Cell Adhesion Protein 1 (VCAM-1)

In one aspect, the assay includes two antibodies AB-1 and AB-2. In certain aspects, AB-1 binds to VCAM-1 at a different epitope site compared to AB-2, i.e., the binding epitope of AB-1 to VCAM-1 is different than the binding epitope of AB-2 to VCAM-1. In one aspect, a human VCAM-1/CD106 antibody from R&D systems can be used for AB-1 (Catalog # BBAS, from monoclonal mouse IgG₁ Clone # BBIG-V1, shown to be specific for human VCAM-1/CD106) and a second, different human VCAM-1/CD106 antibody is used for AB-2 (Catalog# BBA19, from Polyclonal Goat Serum, Detects VCAM-1/CD106, or alternatively, anti-VCAM1 antibody [EPR5047] (ab134047) from abcam which reacts with human VCAM-1). A skilled person will know of other antibodies useful in the present disclosure.

VCAM-1 is a transmembrane cellular adhesion protein that mediates the adhesion of lymphocytes, monocytes, eosinophils, and basophils to vascular endothelium. Upregulation of VCAM-1 in endothelial cells by cytokines occurs as a result of increased gene transcription (e.g., in response to Tumor necrosis factor-alpha (TNFα) and Interleukin-1 (IL-1)). VCAM-1 is encoded by the vascular cell adhesion molecule 1 gene (VCAM1; Entrez GeneID:7412) and is produced after differential splicing of the transcript (Genbank Accession No. NM_001078 (variant 1) or NM_080682 (variant 2)), and processing of the precursor polypeptide splice isoform (Genbank Accession No. NP_001069 (isoform a) or NP_542413 (isoform b)).

The human VCAM1 polypeptide sequence is set forth in, e.g., Genbank Accession No. NP_001069. The human VCAM1 mRNA (coding) sequence is set forth in, e.g., Genbank Accession No. NM_001078. One skilled in the art will appreciate that VCAM1 is also known as VCAM-1, V-CAM1, INCAM-100, CD antigen 106, cluster of differentiation 106, and CD106.

In certain aspects, the methods described herein are used to measure and/or detect VCAM-1. In certain aspects, the concentration or level of VCAM-1 is measured. In certain aspects, the biological sample in which VCAM-1 is measured is whole blood.

In certain aspects, the normal control concentration of VCAM-1 or reference value is about 100 to about 500 ng/mL. In certain aspect, the normal amount of VCAM-1 is about 100 ng/mL, 110 ng/mL, 120 ng/mL, 130 ng/mL, 140 ng/mL, 150 ng/mL, 160 ng/mL, 170 ng/mL, 180 ng/mL, 190 ng/mL, 200 ng/mL, 210 ng/mL, 220 ng/mL, 230 ng/mL, 240 ng/mL, 250 ng/mL, 260 ng/mL, 270 ng/mL, 280 ng/mL, 290 ng/mL, 300 ng/mL, 310 ng/mL, 320 ng/mL, 330 ng/mL, 340 ng/mL, 350 ng/mL, 360 ng/mL, 370 ng/mL, 380 ng/mL, 390 ng/mL, 400 ng/mL, 410 ng/mL, 420 ng/mL, 430 ng/mL, 440 ng/mL, 450 ng/mL, 460 ng/mL, 470 ng/mL, 480 ng/mL, 490 ng/mL, and 500 ng/mL.

In certain aspects, the concentration of VCAM-1 in the biological sample is deemed elevated when it is at least 10% to about 60% greater than the normal control concentration of VCAM-1. In certain aspects, the concentration of VCAM-1 in the biological sample is deemed elevated when it is at least about 10%, 15%, 20%, 25%, 30%, 35%, 40%, 45%, 50%, 55%, and/or 60% or greater than the normal control concentration of VCAM-1. In certain aspects, the concentration of VCAM-1 in the biological sample is deemed elevated when it is at least 550 ng/mL. In some embodiments, the concentration of VCAM-1 in the biological sample is deemed elevated when it is at least 650 ng/mL. In some embodiments, the concentration of VCAM-1 in the biological sample is elevated when it is 650 ng/mL to 1500 ng/mL.

In certain aspects, the methods herein can be used to discriminate between nonalcoholic fatty liver (NAFL) and nonalcoholic steatohepatitis (NASH), by measuring a quantity of VCAM-1 contained in blood collected from a subject; and determining that the subject is affected with or possibly affected with NASH in a case that the quantity of VCAM-1 is elevated or larger than a reference value.

In certain aspects, the methods herein can be used to determine the presence of fibrosis such as hepatic fibrosis by measuring a quantity of VCAM-1 contained in blood collected from a subject; and determining that the subject has or possibly has a symptom of hepatic fibrosis in a case that the quantity of VCAM-1 is elevated or larger than a reference value.

In certain aspects, the method herein can be used to determine a degree of progression of a symptom of nonalcoholic fatty liver disease (NAFLD), by measuring a quantity of VCAM-1 contained in blood collected from a subject if the quantity of VCAM-1 is larger than a reference value.

In certain aspects, the methods can be used to determine the presence or degree of progression of a symptom of NAFLD, NAFL or NASH. The larger the value of VCAM-1, then it is determined that a subject has a possible higher degree of progression of a symptom of NAFLD, NAFL or NASH. Alternatively, it may also be determined that the application of the therapeutic drug is possibly effective in a case that the value after the application of a therapeutic drug is lower than the index value before the application.

The methods of the present embodiment can also be used as a method for determining the degree of progression of a symptom of a hepatic disease. The degree of progression of symptoms of hepatic diseases that has been so far determined based on pathological findings by a liver biopsy can be determined by a non-invasive method. Examples of the hepatic disease include NAFLD, alcoholic liver disorders, chronic hepatitis and drug-induced liver disorders. Examples of the degree of progression of a symptom of a hepatic disease include the degree of progression of hepatic fibrosis, the degree of progression of hepatic inflammation, the degree of progression of hepatocellular ballooning degeneration, the degree of progression of NAFLD activity and hepatocellular necrosis. The hepatic fibrosis is a symptom found in, for example, NAFLD, alcoholic liver disorders, chronic hepatitis and drug-induced liver disorders. The hepatic inflammation, particularly liver lobular inflammation, is a symptom found in, for example, NAFLD, alcoholic liver disorders, chronic hepatitis and drug-induced liver disorders. The hepatocellular ballooning degeneration is a symptom found in, for example, NAFLD, alcoholic liver disorders and chronic hepatitis. The hepatocellular necrosis is a symptom found in, for example, NAFLD, alcoholic liver disorders, chronic hepatitis and drug-induced liver disorders.

### 2. Calprotectin

Calprotectin used in accordance with the presently-disclosed subject matter is a protein heterodimer, including an S100A8 polypeptide, and an S100A9 polypeptide.

S100A8 polypeptides include, H. sapiens S100A8 (Accession Nos: NM-002964, NP-002955; 5.NM_002964.5→NP_002955.2 protein S100-A8 isoform d). S100A8 polypeptides include SEQ ID NO:2.

S100A9 polypeptides include, for example, H. sapiens S100A9 (Accession Nos: NM-002965, NP-002956; NM_002965.4→NP_002956.1 protein S100-A9). S100A9 polypeptides include SEQ ID NO: 3.

S100A8 and S100A9 are small calcium-binding proteins that are highly expressed in neutrophil and monocyte cytosol and are found at high levels in the extracellular milieu during inflammatory conditions. The proteins S100A8 and S100A9 form a heterodimer called calprotectin. S100 calcium-binding protein A8 (S100A8) is a protein that in humans is encoded by the S100A8 gene. It is also known as calgranulin A. S100 calcium-binding protein A9 (S100A9) also known as migration inhibitory factor-related protein 14 (MRP14) or calgranulin B, is a protein that in humans is encoded by the S100A9 gene.

In one embodiment, the present disclosure provides a method for determining inflammation of the gut or gastrointestinal tract, comprising:
contacting the sample with a first antibody having a first binding epitope specific to calprotectin, wherein the first antibody is labeled with a donor fluorophore;
contacting the sample with a second antibody having a second binding epitope specific to calprotectin, wherein the second antibody is labeled with an acceptor fluorophore;
incubating the sample for a time sufficient to obtain a dual labeled calprotectin; and
exciting the sample having the dual labeled calprotectin using a light source to detect a fluorescent emission signal associated with fluorescence resonance energy transfer (FRET).

Fecal calprotectin is useful in differentiating between IBD (Inflammatory Bowel Disease) and IBS (Irritable Bowel Syndrome). Typically, IBD (e.g. Crohn's Disease (CD) or Ulcerative Colitis (UC)) has accompanying inflammation whereas IBS does not have inflammation. A higher than normal level of calprotectin indicates inflammation and thus can be used to differentiate between IBD and IBS.

In certain aspects, the concertation amount of calprotectin is in a range of about 10 μg/g to about 800 μg/g. In certain aspects, the range is about 10 μg/g to about 60 μg/g such as about 0, 5, 10, 15, 20, 25, 30, 35, 40, 45, 50, 55, and/or 60 μg/g. In certain aspects, a range of about 10 μg/g to about 60 μg/g is considered normal or healthy.

In other instances, the concentration amount of calprotectin is in a range of about 10 μg/g to about 100 μg/g, such as 10, 15, 20, 25, 30, 35, 40, 45, 50, 55, 60, 65, 70, 75, 80, 85, 90, 95, and/or 100, which is considered normal or healthy.

In certain instances, a number about 60 μg/g or about 100 μg/g is considered elevated and abnormal. A concentration of calprotectin in a range of about 100 μg/g to about 800 μg/g, such as 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, 395, 400, 405, 410, 415, 420, 425, 430, 435, 440, 445, 450, 455, 460, 465, 470, 475, 480, 485, 490, 495, 500, 505, 510, 515, 520, 525, 530, 535, 540, 545, 550, 555, 560, 565, 570, 575, 580, 585, 590, 595, 600, 605, 610, 615, 620, 625, 630, 635, 640, 645, 650, 655, 660, 665, 670, 675, 680, 685, 690, 695, 700, 705, 710, 715, 720, 725, 730, 735, 740, 745, 750, 755, 760, 765, 770, 775, 780, 785, 790, 795, and/or 800 is considered abnormal.

### 3. Cryptates as FRET Donors

In certain aspects, the terbium cryptate molecule “Lumi4-Tb” from Lumiphore, marketed by Cisbio bioassays is used as the cryptate donor. The terbium cryptate “Lumi4-Tb” having the formula below, which can be coupled to an antibody by a reactive group, in this case, for example, an NHS ester:

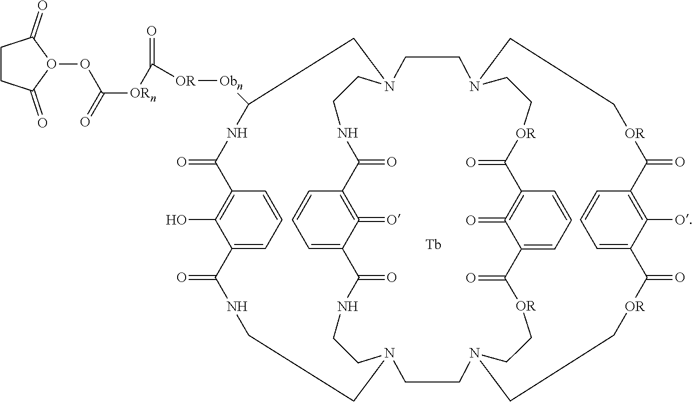

An activated ester (an NHS ester) can react with a primary amine on an antibody to make a stable amide bond. A maleimide on the cryptate and a thiol on the antibody can react together and make a thioether. Alkyl halides react with amines and thiols to make alkylamines and thioethers, respectively. Any derivative providing a reactive moiety that can be conjugated to an antibody can be utilized herein.

In certain other aspects, cryptates disclosed in WO2015157057, titled “Macrocycles” are suitable for use in the present disclosure. This publication contains cryptate molecules useful for labeling biomolecules. As disclosed therein, certain of the cryptates have the structure:

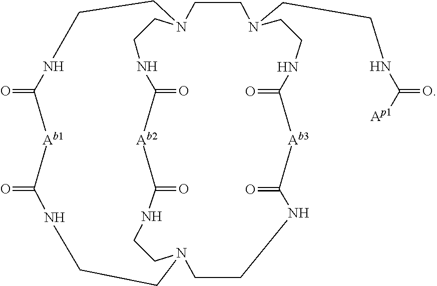

In certain other aspects, a terbium cryptate useful in the present disclosure is shown below:

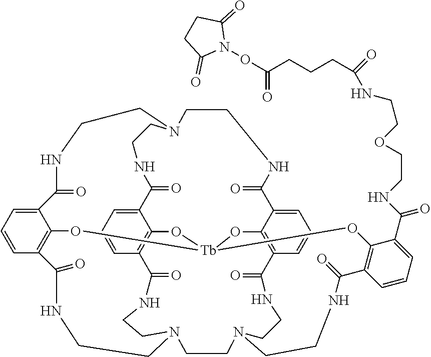

In certain aspects, the cryptates that are useful in the present invention are disclosed in WO 2018/130988, published Jul. 19, 2018. As disclosed therein, the compounds of Formula I are useful as FRET donors in the present disclosure:

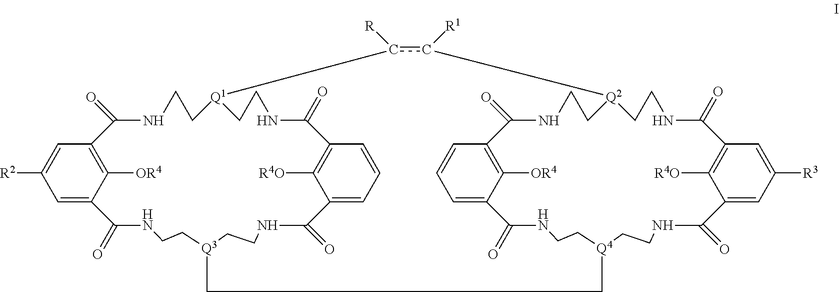

wherein when the dotted line is present, R and R¹ are each independently selected from the group consisting of hydrogen, halogen, hydroxyl, alkyl optionally substituted with one or more halogen atoms, carboxyl, alkoxycarbonyl, amido, sulfonato, alkoxycarbonylalkyl or alkylcarbonylalkoxy or alternatively, R and R¹ join to form an optionally substituted cyclopropyl group wherein the dotted bond is absent;

R² and R³ are each independently a member selected from the group consisting of hydrogen, halogen, SO₃H, —SO₂—X, wherein X is a halogen, optionally substituted alkyl, optionally substituted aryl, optionally substituted alkenyl, optionally substituted alkynyl, optionally substituted cycloalkyl, or an activated group that can be linked to a biomolecule, wherein the activated group is a member selected from the group consisting of a halogen, an activated ester, an activated acyl, optionally substituted alkylsulfonate ester, optionally substituted arylsulfonate ester, amino, formyl, glycidyl, halo, haloacetamidyl, haloalkyl, hydrazinyl, imido ester, isocyanato, isothiocyanato, maleimidyl, mercapto, alkynyl, hydroxyl, alkoxy, amino, cyano, carboxyl, alkoxycarbonyl, amido, sulfonato, alkoxycarbonylalkyl, cyclic anhydride, alkoxyalkyl, a water solubilizing group or L;

R⁴ are each independently a hydrogen, C₁-C₆ alkyl, or alternatively, 3 of the R⁴ groups are absent and the resulting oxides are chelated to a lanthanide cation; and

Q¹-Q⁴ are each independently a member selected from the group of carbon or nitrogen.

### 4. FRET Acceptors

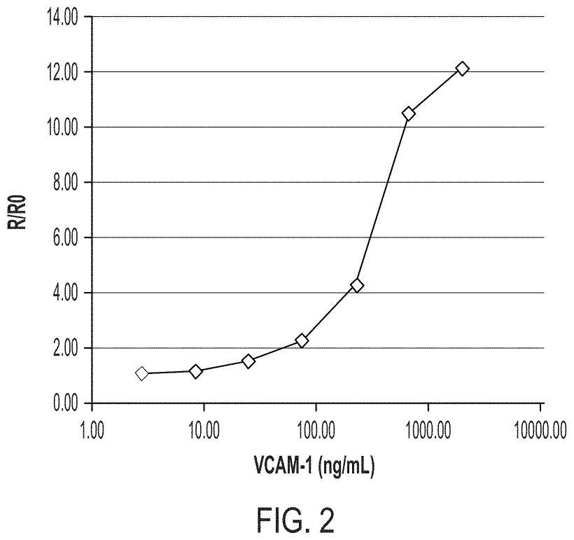

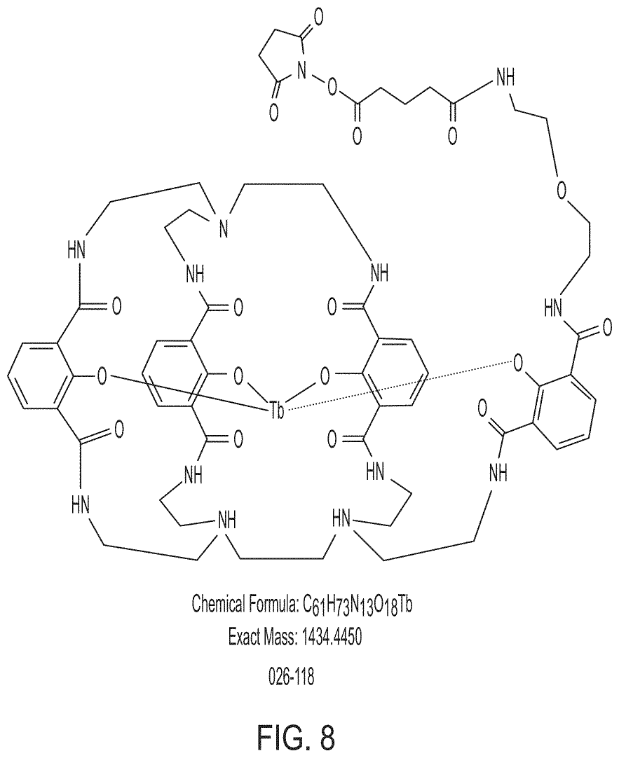

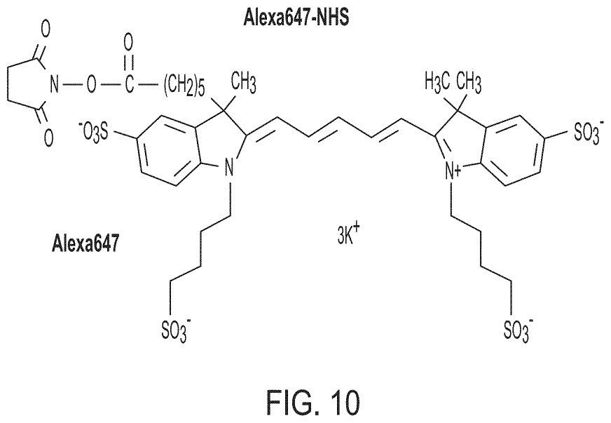

In order to detect a FRET signal, a FRET acceptor is required. The FRET acceptor has an excitation wavelength that overlaps with an emission wavelength of the FRET donor. The FRET signal of the acceptor is proportional to the concentration level of VCAM-1 present in the sample, such as a patient's blood sample as interpolated from a known amount of calibrators i.e., a standard curve (FIG. 2). A cryptate donor can be used to label the first antibody AB-1 (FIG. 8). Lumi4 has 3 spectrally distinct peaks, at 490, 550 and 620 nm, which can be used for energy transfer (FIG. 9). An acceptor can be used to label the second antibody AB-2. The acceptor molecules that can be used include, but are not limited to, fluorescein-like (green zone) acceptor, Cy5, DY-647, Alexa Fluor 488, Alexa Fluor 546, Allophycocyanin (APC), and Phycoeruythrin (PE) and Alexa Fluor 647 (FIG. 10). Donor and acceptor fluorophores can be conjugated using a primary amine on an antibody.

In certain aspects of the embodiments, the assay uses a donor fluorophore consisting of terbium bound within a cryptate. The terbium cryptate can be excited with a 365 nm UV LED. The terbium cryptate emits at four (4) wavelengths within the visible region. In one aspect, the assay uses the lowest donor emission energy peak of 620 nm as the donor signal within the assay. In certain aspects, the acceptor fluorophore, when in very close proximity, is excited by the highest energy terbium cryptate emission peak of 490 nm causing light emission at 520 nm. Both the 620 nm and 520 nm emission wavelengths are measured independently in a device or instrument and results can be reported as RFU ratio 620/520.

Other acceptors include, but are not limited to, cyanin derivatives, D2, CYS, fluorescein, coumarin, rhodamine, carbopyronine, oxazine and its analogs, Alexa Fluor fluorophores, Crystal violet, perylene bisimide fluorophores, squaraine fluorophores, boron dipyrromethene derivatives, NBD (nitrobenzoxadiazole) and its derivatives, DABCYL (4-((4-(dimethylamino)phenyl)azo)benzoic acid).

In one aspect, fluorescence can be characterized by wavelength, intensity, lifetime, polarization or a combination thereof.

### 5. Antibodies

In one aspect, a human VCAM-1/CD106 antibody from R&D systems can be used for AB-1 (Catalog # BBAS, from monoclonal mouse IgG₁ Clone # BBIG-V1, shown to be specific for human VCAM-1/CD106) and a different human VCAM-1/CD106 antibody for AB-2 (Catalog# BBA19, from Polyclonal Goat Serum, Detects VCAM-1/CD106 or Anti-VCAM1 antibody [EPR5047] (ab134047) from abcam which reacts with human), or vice versa. Those of skill in the art will know of other antibodies suitable for use in the present disclosure.

In certain aspects, an activated ester (an NHS ester) of the donor or acceptor can react with a primary amine on an antibody to make a stable amide bond. For example, a maleimide on the cryptate or the acceptor (e.g., Alexa 647) and a thiol on the antibody can react together and make a thioether. Alkyl halides react with amines and thiols to make alkylamines and thioethers, respectively. Any derivative providing a reactive moiety that can be conjugated to an antibody can be utilized herein to make the first antibody labeled with a donor fluorophore specific for VCAM-1, as well as, the second antibody labeled with an acceptor fluorophore specific for VCAM-1.

The methods herein for detecting the presence or level of VCAM-1 can use a variety of samples, which include a tissue sample, blood, biopsy, serum, plasma, saliva, urine, or stool sample.

### 6. Production of Antibodies

The generation and selection of antibodies not already commercially available can be accomplished several ways. For example, one way is to express and/or purify a polypeptide of interest (i.e., antigen) using protein expression and purification methods known in the art, while another way is to synthesize the polypeptide of interest using solid phase peptide synthesis methods known in the art. See, e.g., Guide to Protein Purification, Murray P. Deutcher, ed., Meth. Enzymol., Vol. 182 (1990); Solid Phase Peptide Synthesis, Greg B. Fields, ed., Meth. Enzymol., Vol. 289 (1997); Kiso et al., Chem. Pharm. Bull., 38:1192-99 (1990); Mostafavi et al., Biomed. Pept. Proteins Nucleic Acids, 1:255-60, (1995); and Fujiwara et al., Chem. Pharm. Bull., 44:1326-31 (1996). The purified or synthesized polypeptide can then be injected, for example, into mice or rabbits, to generate polyclonal or monoclonal antibodies. One skilled in the art will recognize that many procedures are available for the production of antibodies, for example, as described in Antibodies, A Laboratory Manual, Harlow and Lane, Eds., Cold Spring Harbor Laboratory, Cold Spring Harbor, N.Y. (1988). One skilled in the art will also appreciate that binding fragments or Fab fragments which mimic antibodies can also be prepared from genetic information by various procedures (see, e.g., Antibody Engineering: A Practical Approach, Borrebaeck, Ed., Oxford University Press, Oxford (1995); and Huse et al., J. Immunol., 149:3914-3920 (1992)).

In addition, numerous publications have reported the use of phage display technology to produce and screen libraries of polypeptides for binding to a selected target antigen (see, e.g, Cwirla et al., Proc. Natl. Acad. Sci. USA, 87:6378-6382 (1990); Devlin et al., Science, 249:404-406 (1990); Scott et al., Science, 249:386-388 (1990); and Ladner et al., U.S. Pat. No. 5,571,698). A basic concept of phage display methods is the establishment of a physical association between a polypeptide encoded by the phage DNA and a target antigen. This physical association is provided by the phage particle, which displays a polypeptide as part of a capsid enclosing the phage genome which encodes the polypeptide. The establishment of a physical association between polypeptides and their genetic material allows simultaneous mass screening of very large numbers of phage bearing different polypeptides. Phage displaying a polypeptide with affinity to a target antigen bind to the target antigen and these phage are enriched by affinity screening to the target antigen. The identity of polypeptides displayed from these phage can be determined from their respective genomes. Using these methods, a polypeptide identified as having a binding affinity for a desired target antigen can then be synthesized in bulk by conventional means (see, e.g., U.S. Pat. No. 6,057,098).

The antibodies that are generated by these methods can then be selected by first screening for affinity and specificity with the purified polypeptide antigen of interest and, if required, comparing the results to the affinity and specificity of the antibodies with other polypeptide antigens that are desired to be excluded from binding. The screening procedure can involve immobilization of the purified polypeptide antigens in separate wells of microtiter plates. The solution containing a potential antibody or group of antibodies is then placed into the respective microtiter wells and incubated for about 30 minutes to 2 hours. The microtiter wells are then washed and a labeled secondary antibody (e.g., an anti-mouse antibody conjugated to alkaline phosphatase if the raised antibodies are mouse antibodies) is added to the wells and incubated for about 30 minutes and then washed. Substrate is added to the wells and a color reaction will appear where antibody to the immobilized polypeptide antigen is present.

The antibodies so identified can then be further analyzed for affinity and specificity. In the development of immunoassays for a target protein (VCAM-1), the purified target protein acts as a standard with which to judge the sensitivity and specificity of the immunoassay using the antibodies that have been selected. Because the binding affinity of various antibodies may differ, e.g., certain antibody combinations may interfere with one another sterically, assay performance of an antibody may be a more important measure than absolute affinity and specificity of that antibody.

Those skilled in the art will recognize that many approaches can be taken in producing antibodies or binding fragments and screening and selecting for affinity and specificity for the various polypeptides of interest, but these approaches do not change the scope of the present invention.

A. Polyclonal Antibodies

Polyclonal antibodies are preferably raised in animals by multiple subcutaneous (sc) or intraperitoneal (ip) injections of a polypeptide of interest and an adjuvant. It may be useful to conjugate the polypeptide of interest to a protein carrier that is immunogenic in the species to be immunized, such as, e.g., keyhole limpet hemocyanin, serum albumin, bovine thyroglobulin, or soybean trypsin inhibitor using a bifunctional or derivatizing agent. Non-limiting examples of bifunctional or derivatizing agents include maleimidobenzoyl sulfosuccinimide ester (conjugation through cysteine residues), N-hydroxysuccinimide (conjugation through lysine residues), glutaraldehyde, succinic anhydride, SOC12, and R₁N═C═NR, wherein R and R₁ are different alkyl groups.

Animals are immunized against the polypeptide of interest or an immunogenic conjugate or derivative thereof by combining, e.g., 100 μg (for rabbits) or 5 μg (for mice) of the antigen or conjugate with 3 volumes of Freund's complete adjuvant and injecting the solution intradermally at multiple sites. One month later, the animals are boosted with about ⅕ to 1/10 the original amount of polypeptide or conjugate in Freund's incomplete adjuvant by subcutaneous injection at multiple sites. Seven to fourteen days later, the animals are bled and the serum is assayed for antibody titer. Animals are typically boosted until the titer plateaus. Preferably, the animal is boosted with the conjugate of the same polypeptide, but conjugation to a different immunogenic protein and/or through a different cross-linking reagent may be used. Conjugates can also be made in recombinant cell culture as fusion proteins. In certain instances, aggregating agents such as alum can be used to enhance the immune response.

B. Monoclonal Antibodies

Monoclonal antibodies are generally obtained from a population of substantially homogeneous antibodies, i.e., the individual antibodies comprising the population are identical except for possible naturally-occurring mutations that may be present in minor amounts. Thus, the modifier “monoclonal” indicates the character of the antibody as not being a mixture of discrete antibodies. For example, monoclonal antibodies can be made using the hybridoma method described by Kohler et al., Nature, 256:495 (1975) or by any recombinant DNA method known in the art (see, e.g., U.S. Pat. No. 4,816,567).

In the hybridoma method, a mouse or other appropriate host animal (e.g., hamster) is immunized as described above to elicit lymphocytes that produce or are capable of producing antibodies which specifically bind to the polypeptide of interest used for immunization. Alternatively, lymphocytes are immunized in vitro. The immunized lymphocytes are then fused with myeloma cells using a suitable fusing agent, such as polyethylene glycol, to form hybridoma cells (see, e.g., Goding, Monoclonal Antibodies: Principles and Practice, Academic Press, pp. 59-103 (1986)). The hybridoma cells thus prepared are seeded and grown in a suitable culture medium that preferably contains one or more substances which inhibit the growth or survival of the unfused, parental myeloma cells. For example, if the parental myeloma cells lack the enzyme hypoxanthine guanine phosphoribosyl transferase (HGPRT), the culture medium for the hybridoma cells will typically include hypoxanthine, aminopterin, and thymidine (HAT medium), which prevent the growth of HGPRT-deficient cells.

Preferred myeloma cells are those that fuse efficiently, support stable high-level production of antibody by the selected antibody-producing cells, and/or are sensitive to a medium such as HAT medium. Examples of such preferred myeloma cell lines for the production of human monoclonal antibodies include, but are not limited to, murine myeloma lines such as those derived from MOPC-21 and MPC-11 mouse tumors (available from the Salk Institute Cell Distribution Center; San Diego, Calif.), SP-2 or X63-Ag8-653 cells (available from the American Type Culture Collection; Rockville, Md., and human myeloma or mouse-human heteromyeloma cell lines (see, e.g., Kozbor, J. Immunol., 133:3001 (1984); and Brodeur et al., Monoclonal Antibody Production Techniques and Applications, Marcel Dekker, Inc., New York, pp. 51-63 (1987)).

The culture medium in which hybridoma cells are growing can be assayed for the production of monoclonal antibodies directed against the polypeptide of interest. Preferably, the binding specificity of monoclonal antibodies produced by hybridoma cells is determined by immunoprecipitation or by an in vitro binding assay, such as a radioimmunoassay (RIA) or an enzyme-linked immunoabsorbent assay (ELISA). The binding affinity of monoclonal antibodies can be determined using, e.g., the Scatchard analysis of Munson et al., Anal. Biochem., 107:220 (1980).

After hybridoma cells are identified that produce antibodies of the desired specificity, affinity, and/or activity, the clones may be subcloned by limiting dilution procedures and grown by standard methods (see, e.g., Goding, Monoclonal Antibodies: Principles and Practice, Academic Press, pp. 59-103 (1986)). Suitable culture media for this purpose include, for example, D-MEM or RPMI-1640 medium. In addition, the hybridoma cells may be grown in vivo as ascites tumors in an animal. The monoclonal antibodies secreted by the subclones can be separated from the culture medium, ascites fluid, or serum by conventional antibody purification procedures such as, for example, protein A-Sepharose, hydroxylapatite chromatography, gel electrophoresis, dialysis, or affinity chromatography.

DNA encoding the monoclonal antibodies can be readily isolated and sequenced using conventional procedures (e.g., by using oligonucleotide probes that are capable of binding specifically to genes encoding the heavy and light chains of murine antibodies). The hybridoma cells serve as a preferred source of such DNA. Once isolated, the DNA may be placed into expression vectors, which are then transfected into host cells such as E. coli cells, simian COS cells, Chinese Hamster Ovary (CHO) cells, or myeloma cells that do not otherwise produce antibody, to induce the synthesis of monoclonal antibodies in the recombinant host cells. See, e.g., Skerra et al., Curr. Opin. Immunol., 5:256-262 (1993); and Pluckthun, Immunol Rev., 130:151-188 (1992). The DNA can also be modified, for example, by substituting the coding sequence for human heavy chain and light chain constant domains in place of the homologous murine sequences (see, e.g., U.S. Pat. No. 4,816,567; and Morrison et al., Proc. Natl. Acad. Sci. USA, 81:6851 (1984)), or by covalently joining to the immunoglobulin coding sequence all or part of the coding sequence for a non-immunoglobulin polypeptide.

In a further embodiment, monoclonal antibodies or antibody fragments can be isolated from antibody phage libraries generated using the techniques described in, for example, McCafferty et al., Nature, 348:552-554 (1990); Clackson et al., Nature, 352:624-628 (1991); and Marks et al., J. Mol. Biol., 222:581-597 (1991). The production of high affinity (nM range) human monoclonal antibodies by chain shuffling is described in Marks et al., BioTechnology, 10:779-783 (1992). The use of combinatorial infection and in vivo recombination as a strategy for constructing very large phage libraries is described in Waterhouse et al., Nuc. Acids Res., 21:2265-2266 (1993). Thus, these techniques are viable alternatives to traditional monoclonal antibody hybridoma methods for the generation of monoclonal antibodies.Human Antibodies

As an alternative to humanization, human antibodies can be generated. In some embodiments, transgenic animals (e.g., mice) can be produced that are capable, upon immunization, of producing a full repertoire of human antibodies in the absence of endogenous immunoglobulin production. For example, it has been described that the homozygous deletion of the antibody heavy-chain joining region (JH) gene in chimeric and germ-line mutant mice results in complete inhibition of endogenous antibody production. Transfer of the human germ-line immunoglobulin gene array in such germ-line mutant mice will result in the production of human antibodies upon antigen challenge. See, e.g., Jakobovits et al., Proc. Natl. Acad. Sci. USA, 90:2551 (1993); Jakobovits et al., Nature, 362:255-258 (1993); Bruggermann et al., Year in Immun., 7:33 (1993); and U.S. Pat. Nos. 5,591,669, 5,589,369, and 5,545,807.

Alternatively, phage display technology (see, e.g., McCafferty et al., Nature, 348:552-553 (1990)) can be used to produce human antibodies and antibody fragments in vitro, using immunoglobulin variable (V) domain gene repertoires from unimmunized donors. According to this technique, antibody V domain genes are cloned in-frame into either a major or minor coat protein gene of a filamentous bacteriophage, such as M13 or fd, and displayed as functional antibody fragments on the surface of the phage particle. Because the filamentous particle contains a single-stranded DNA copy of the phage genome, selections based on the functional properties of the antibody also result in selection of the gene encoding the antibody exhibiting those properties. Thus, the phage mimics some of the properties of the B cell. Phage display can be performed in a variety of formats as described in, e.g., Johnson et al., Curr. Opin. Struct. Biol., 3:564-571 (1993). Several sources of V-gene segments can be used for phage display. See, e.g., Clackson et al., Nature, 352:624-628 (1991). A repertoire of V genes from unimmunized human donors can be constructed and antibodies to a diverse array of antigens (including self-antigens) can be isolated essentially following the techniques described in Marks et al., J. Mol. Biol., 222:581-597 (1991); Griffith et al., EMBO 1, 12:725-734 (1993); and U.S. Pat. Nos. 5,565,332 and 5,573,905.

In certain instances, human antibodies can be generated by in vitro activated B cells as described in, e.g., U.S. Pat. Nos. 5,567,610 and 5,229,275.

C. Antibody Fragments

Various techniques have been developed for the production of antibody fragments.

Traditionally, these fragments were derived via proteolytic digestion of intact antibodies (see, e.g., Morimoto et al., J. Biochem. Biophys. Meth., 24:107-117 (1992); and Brennan et al., Science, 229:81 (1985)). However, these fragments can now be produced directly using recombinant host cells. For example, the antibody fragments can be isolated from the antibody phage libraries discussed above. Alternatively, Fab′-SH fragments can be directly recovered from E. coli cells and chemically coupled to form F(ab′)₂ fragments (see, e.g., Carter et al., BioTechnology, 10:163-167 (1992)). According to another approach, F(ab′)₂ fragments can be isolated directly from recombinant host cell culture. Other techniques for the production of antibody fragments will be apparent to those skilled in the art. In other embodiments, the antibody of choice is a single chain Fv fragment (scFv). See, e.g., PCT Publication No. WO 93/16185; and U.S. Pat. Nos. 5,571,894 and 5,587,458. The antibody fragment may also be a linear antibody as described, e.g., in U.S. Pat. No. 5,641,870. Such linear antibody fragments may be monospecific or bispecific.

D. Bispecific Antibodies

Bispecific antibodies are antibodies that have binding specificities for at least two different epitopes. Exemplary bispecific antibodies may bind to two different epitopes of the same polypeptide of interest. Other bispecific antibodies may combine a binding site for the polypeptide of interest with binding site(s) for one or more additional antigens. Bispecific antibodies can be prepared as full-length antibodies or antibody fragments (e.g., F(ab′)₂ bispecific antibodies).

Methods for making bispecific antibodies are known in the art. Traditional production of full-length bispecific antibodies is based on the co-expression of two immunoglobulin heavy chain-light chain pairs, where the two chains have different specificities (see, e.g., Millstein et al., Nature, 305:537-539 (1983)). Because of the random assortment of immunoglobulin heavy and light chains, these hybridomas (quadromas) produce a potential mixture of 10 different antibody molecules, of which only one has the correct bispecific structure. Purification of the correct molecule is usually performed by affinity chromatography. Similar procedures are disclosed in PCT Publication No. WO 93/08829 and Traunecker et al., EMBO J., 10:3655-3659 (1991).

According to a different approach, antibody variable domains with the desired binding specificities (antibody-antigen combining sites) are fused to immunoglobulin constant domain sequences. The fusion preferably is with an immunoglobulin heavy chain constant domain, comprising at least part of the hinge, CH2, and CH3 regions. It is preferred to have the first heavy chain constant region (CH1) containing the site necessary for light chain binding present in at least one of the fusions. DNA encoding the immunoglobulin heavy chain fusions and, if desired, the immunoglobulin light chain, are inserted into separate expression vectors, and are co-transfected into a suitable host organism. This provides for great flexibility in adjusting the mutual proportions of the three polypeptide fragments in embodiments when unequal ratios of the three polypeptide chains used in the construction provide the optimum yields. It is, however, possible to insert the coding sequences for two or all three polypeptide chains into one expression vector when the expression of at least two polypeptide chains in equal ratios results in high yields or when the ratios are of no particular significance.

In a preferred embodiment of this approach, the bispecific antibodies are composed of a hybrid immunoglobulin heavy chain with a first binding specificity in one arm, and a hybrid immunoglobulin heavy chain-light chain pair (providing a second binding specificity) in the other arm. This asymmetric structure facilitates the separation of the desired bispecific compound from unwanted immunoglobulin chain combinations, as the presence of an immunoglobulin light chain in only one half of the bispecific molecule provides for a facile way of separation. See, e.g., PCT Publication No. WO 94/04690 and Suresh et al., Meth. Enzymol., 121:210 (1986).

According to another approach described in U.S. Pat. No. 5,731,168, the interface between a pair of antibody molecules can be engineered to maximize the percentage of heterodimers which are recovered from recombinant cell culture. The preferred interface comprises at least a part of the CH3 domain of an antibody constant domain. In this method, one or more small amino acid side-chains from the interface of the first antibody molecule are replaced with larger side chains (e.g., tyrosine or tryptophan). Compensatory “cavities” of identical or similar size to the large side-chain(s) are created on the interface of the second antibody molecule by replacing large amino acid side-chains with smaller ones (e.g., alanine or threonine). This provides a mechanism for increasing the yield of the heterodimer over other unwanted end-products such as homodimers.

Bispecific antibodies include cross-linked or “heteroconjugate” antibodies. For example, one of the antibodies in the heteroconjugate can be coupled to avidin, the other to biotin. Heteroconjugate antibodies can be made using any convenient cross-linking method. Suitable cross-linking agents and techniques are well-known in the art, and are disclosed in, e.g., U.S. Pat. No. 4,676,980.

Suitable techniques for generating bispecific antibodies from antibody fragments are also known in the art. For example, bispecific antibodies can be prepared using chemical linkage. In certain instances, bispecific antibodies can be generated by a procedure in which intact antibodies are proteolytically cleaved to generate F(ab′)₂ fragments (see, e.g., Brennan et al., Science, 229:81 (1985)). These fragments are reduced in the presence of the dithiol complexing agent sodium arsenite to stabilize vicinal dithiols and prevent intermolecular disulfide formation. The Fab′ fragments generated are then converted to thionitrobenzoate (TNB) derivatives. One of the Fab′-TNB derivatives is then reconverted to the Fab′-thiol by reduction with mercaptoethylamine and is mixed with an equimolar amount of the other Fab′-TNB derivative to form the bispecific antibody.

In some embodiments, Fab′-SH fragments can be directly recovered from E. coli and chemically coupled to form bispecific antibodies. For example, a fully humanized bispecific antibody F(ab′)₂ molecule can be produced by the methods described in Shalaby et al., J. Exp. Med., 175: 217-225 (1992). Each Fab′ fragment was separately secreted from E. coli and subjected to directed chemical coupling in vitro to form the bispecific antibody.

Various techniques for making and isolating bispecific antibody fragments directly from recombinant cell culture have also been described. For example, bispecific antibodies have been produced using leucine zippers. See, e.g., Kostelny et al., J. Immunol., 148:1547-1553 (1992). The leucine zipper peptides from the Fos and Jun proteins were linked to the Fab′ portions of two different antibodies by gene fusion. The antibody homodimers were reduced at the hinge region to form monomers and then re-oxidized to form the antibody heterodimers. This method can also be utilized for the production of antibody homodimers. The “diabody” technology described by Hollinger et al., Proc. Natl. Acad. Sci. USA, 90:6444-6448 (1993) has provided an alternative mechanism for making bispecific antibody fragments. The fragments comprise a heavy chain variable domain (VH) connected to a light chain variable domain (VL) by a linker which is too short to allow pairing between the two domains on the same chain. Accordingly, the VH and VL domains of one fragment are forced to pair with the complementary VL and VH domains of another fragment, thereby forming two antigen binding sites. Another strategy for making bispecific antibody fragments by the use of single-chain Fv (sFv) dimers is described in Gruber et al., J. Immunol., 152:5368 (1994).

Antibodies with more than two valencies are also contemplated. For example, trispecific antibodies can be prepared. See, e.g., Tutt et al., J. Immunol., 147:60 (1991).

E. Antibody Purification

When using recombinant techniques, antibodies can be produced inside an isolated host cell, in the periplasmic space of a host cell, or directly secreted from a host cell into the medium. If the antibody is produced intracellularly, the particulate debris is first removed, for example, by centrifugation or ultrafiltration. Carter et al., BioTech., 10:163-167 (1992) describes a procedure for isolating antibodies which are secreted into the periplasmic space of E. coli. Briefly, cell paste is thawed in the presence of sodium acetate (pH 3.5), EDTA, and phenylmethylsulfonylfluoride (PMSF) for about 30 min. Cell debris can be removed by centrifugation. Where the antibody is secreted into the medium, supernatants from such expression systems are generally concentrated using a commercially available protein concentration filter, for example, an Amicon or Millipore Pellicon ultrafiltration unit. A protease inhibitor such as PMSF may be included in any of the foregoing steps to inhibit proteolysis and antibiotics may be included to prevent the growth of adventitious contaminants.

The antibody composition prepared from cells can be purified using, for example, hydroxylapatite chromatography, gel electrophoresis, dialysis, and affinity chromatography. The suitability of protein A as an affinity ligand depends on the species and isotype of any immunoglobulin Fc domain that is present in the antibody. Protein A can be used to purify antibodies that are based on human γ1, γ2, or γ4 heavy chains (see, e.g., Lindmark et al., J. Immunol. Meth., 62:1-13 (1983)). Protein G is recommended for all mouse isotypes and for human γ3 (see, e.g., Guss et al., EMBO J., 5:1567-1575 (1986)). The matrix to which the affinity ligand is attached is most often agarose, but other matrices are available. Mechanically stable matrices such as controlled pore glass or poly(styrenedivinyl)benzene allow for faster flow rates and shorter processing times than can be achieved with agarose. Where the antibody comprises a CH3 domain, the Bakerbond ABX™ resin (J. T. Baker; Phillipsburg, N.J.) is useful for purification. Other techniques for protein purification such as fractionation on an ion-exchange column, ethanol precipitation, reverse phase HPLC, chromatography on silica, chromatography on heparin SEPHAROSE™, chromatography on an anion or cation exchange resin (such as a polyaspartic acid column), chromatofocusing, SDS-PAGE, and ammonium sulfate precipitation are also available depending on the antibody to be recovered.

Following any preliminary purification step(s), the mixture comprising the antibody of interest and contaminants may be subjected to low pH hydrophobic interaction chromatography using an elution buffer at a pH between about 2.5-4.5, preferably performed at low salt concentrations (e.g., from about 0-0.25 M salt).

### III. DEVICE

Various instruments and devices are suitable for use in the present disclosure. Many spectrophotometers have the capability to measure fluorescence. Fluorescence is the molecular absorption of light energy at one wavelength and its nearly instantaneous re-emission at another, longer wavelength. Some molecules fluoresce naturally, and others must be modified to fluoresce.

A fluorescence spectrophotometer or fluorometer, fluorospectrometer, or fluorescence spectrometer measures the fluorescent light emitted from a sample at different wavelengths, after illumination with light source such as a xenon flash lamp. Fluorometers can have different channels for measuring differently-colored fluorescent signals (that differ in their wavelengths), such as green and blue, or ultraviolet and blue, channels. In one aspect, a suitable device includes an ability to perform a time-resolved fluorescence resonance energy transfer (FRET) experiment.

Suitable fluorometers can hold samples in different ways, including cuvettes, capillaries, Petri dishes, and microplates. The assays described herein can be performed on any of these types of instruments. In certain aspects, the device has an optional microplate reader, allowing emission scans in up to 384-well plates. Other models suitable for use hold the sample in place using surface tension.

Time-resolved fluorescence (TRF) measurement is similar to fluorescence intensity measurement. One difference, however, is the timing of the excitation/measurement process. When measuring fluorescence intensity, the excitation and emission processes are simultaneous: the light emitted by the sample is measured while excitation is taking place. Even though emission systems are very efficient at removing excitation light before it reaches the detector, the amount of excitation light compared to emission light is such that fluorescent intensity measurements exhibit elevated background signals. The present disclosure offers a solution to this issue. Time resolve FRET relies on the use of specific fluorescent molecules that have the property of emitting over long periods of time (measured in milliseconds) after excitation, when most standard fluorescent dyes (e.g. fluorescein) emit within a few nanoseconds of being excited. As a result, it is possible to excite cryptate lanthanides using a pulsed light source (e.g., Xenon flash lamp or pulsed laser), and measure after the excitation pulse.

As the donor and acceptor fluorescent compounds attached to antibody 1 and 2 move closer together, an energy transfer is caused from the donor compound to the acceptor compound, resulting in a decrease in the fluorescence signal emitted by the donor compound and an increase in the signal emitted by the acceptor compound, and vice-versa. The majority of biological phenomena involving interactions between different partners will therefore be able to be studied by measuring the change in FRET between 2 fluorescent compounds coupled with compounds which will be at a greater or lesser distance, depending on the biological phenomenon in question.

The FRET signal can be measured in different ways: measurement of the fluorescence emitted by the donor alone, by the acceptor alone or by the donor and the acceptor, or measurement of the variation in the polarization of the light emitted in the medium by the acceptor as a result of FRET. One can also include measurement of FRET by observing the variation in the lifetime of the donor, which is facilitated by using a donor with a long fluorescence lifetime, such as rare earth complexes (especially on simple equipment like plate readers). Furthermore, the FRET signal can be measured at a precise instant or at regular intervals, making it possible to study its change over time and thereby to investigate the kinetics of the biological process studied.

In certain aspects, the device disclosed in PCT/IB2019/051213, filed Feb. 14, 2019 is used, which is hereby incorporated by reference. That disclosure in that application generally relates to analyzers that can be used in point-of-care (POC) settings to measure the absorbance and fluorescence of a sample with minimal or no user handling or interaction. The disclosed analyzers provide advantageous features of more rapid and reliable analyses of samples having properties that can be detected with each of these two approaches. For example, it can be beneficial to quantify both the fluorescence and absorbance of a blood sample being subjected to a diagnostic assay. In some analytical workflows, the hematocrit of a blood sample can be quantified with an absorbance assay, while the signal intensities measured in a FRET assay can provide information regarding other components of the blood sample.

One apparatus disclosed in PCT/IB2019/051213 is useful for detecting an emission light from a sample, and absorbance of a transillumination light by the sample, which comprises a first light source configured to emit an excitation light having an excitation wavelength. The apparatus further comprises a second light source configured to transilluminate the sample with the transillumination light. The apparatus further comprises a first light detector configured to detect the excitation light, and a second light detector configured to detect the emission light and the transillumination light. The apparatus further comprises a dichroic mirror configured to (1) epi-illuminate the sample by reflecting at least a portion of the excitation light, (2) transmit at least a portion of the excitation light to the first light detector, (3) transmit at least a portion of the emission light to the second light detector, and (4) transmit at least a portion of the transillumination light to the second light detector.

One suitable cuvette for use in the present disclosure is disclosed in PCT/IB2019/051215, filed Feb. 14, 2019. One of the provided cuvettes comprises a hollow body enclosing an inner chamber having an open chamber top. The cuvette further comprises a lower lid having an inner wall, an outer wall, an open lid top, and an open lid bottom. At least a portion of the lower lid is configured to fit inside the inner chamber proximate to the open chamber top. The lower lid comprises one or more (e.g., two or more) containers connected to the inner wall, wherein each of the containers has an open container top. In certain aspects, the lower lid comprises two or more such containers. The lower lid further comprises a securing means connected to the hollow body. The cuvette further comprises an upper lid wherein at least a portion of the upper lid is configured to fit inside the lower lid proximate to the open lid top

### IV. EXAMPLES

Example 1 illustrates a method of this disclosure detecting the presence and amount of VCAM-1 in a trFRET assay. As shown in FIG. 1, VCAM-1 binds to an anti-VCAM-1 antibody (MAB-1) labeled with a donor fluorophore and a second antibody (MAB-2) labeled with acceptor fluorophore to generate a dual-labeled VCAM-1. The VCAM-1 analyte is in a sample from a patient (e.g., whole blood sample) and it binds to both anti-VCAM-1 antibodies simultaneously in a sandwich assay resulting in a FRET signal.

If one anti-VCAM-1 antibody is labeled with a donor fluorophore and a second anti-VCAM-1 antibody is labeled with an acceptor fluorophore, TR-FRET can occur in the presence of the VCAM-1 antigen (FIG. 1). The increase in FRET signal of the acceptor is proportional to the level of VCAM-1 present in the patient's blood as interpolated from a known amount of VCAM-1 calibrators (FIG. 2). Donor and acceptor fluorophores are conjugated using primary amines on antibodies.

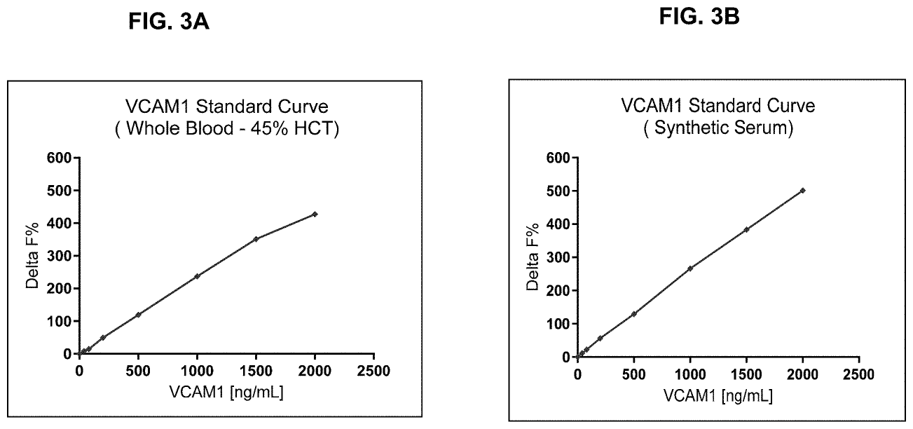

Delta F (signal-background/background %) is used for the comparison of day-to-day runs of the same assay or assays run by different users. It reflects the signal to background of the assay. The negative control plays the role of an internal assay control. The ΔF % data points are tabulated below. FIG. 3A shows the results of the assay using whole blood.

| STDS [ng/mL] | Delta F % |
|---|---|
| 2000 | 427 |
| 1500 | 351 |
| 1000 | 237 |
| 500 | 119 |
| 200 | 49 |
| 80 | 14 |
| 40 | 8 |
| 0 | 0 |

Delta F (signal-background/background %) for synthetic serum is shown in FIG. 3B and tabulated below.

| STDS [ng/mL] | Delta F % |
|---|---|
| 2000 | 501 |
| 1500 | 383 |
| 1000 | 266 |
| 500 | 129 |
| 200 | 56 |
| 80 | 22 |
| 40 | 11 |
| 0 | 0 |

Example 2 illustrates collection of stools and preparation of stool extracts and standard curves for calprotectin.

Stools are collected in plastic containers and immediately frozen below −20° C. In order to prepare extracts, the stools are thawed and 5 grams aliquots are collected, suspended with 10 ml of fecal extraction buffer and homogenized on ice for one minute at 20000 rpm, using an mechanical homogenizer. The temperature is maintained between 20° C. and 23° C. during this procedure. The homogenates are centrifuged at 45000 g for 20 minutes at 4° C. and the top halves of the supernatants are pipetted off and can be used.

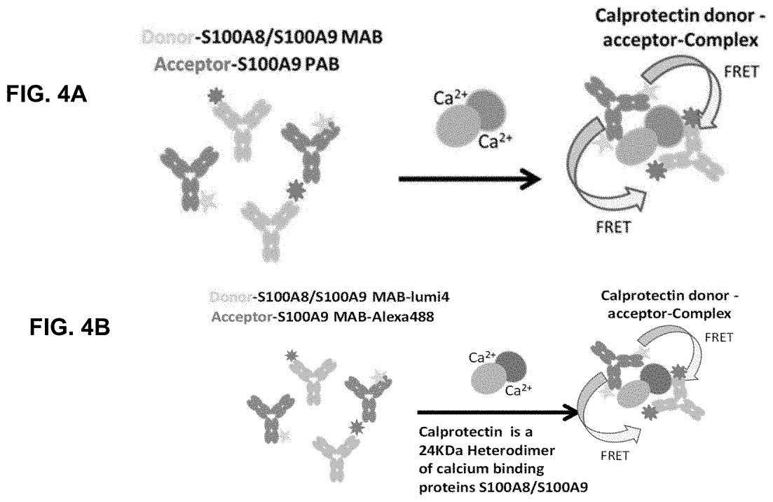

This examples illustrates a method of this disclosure detecting the presence and amount of calprotectin in a trFRET assay. As shown in FIGS. 4A-B, calprotectin binds to an anti-calprotectin antibody (MAB-1) labeled with a donor fluorophore and a second antibody (MAB-2) labeled with acceptor fluorophore. The calprotectin analyte is in a sample from a patient (i.e., fecal sample, prepared as above) and it binds to both anti-calprotectin antibodies simultaneously resulting in a dual labeled calprotectin. After light excitation, a FRET signal occurs and is detected.

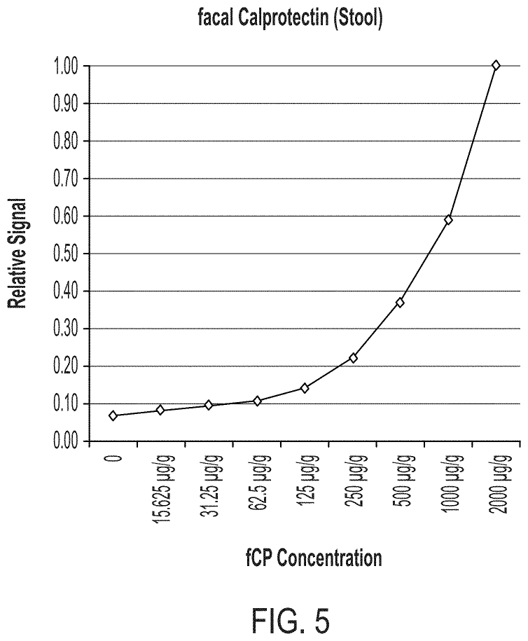

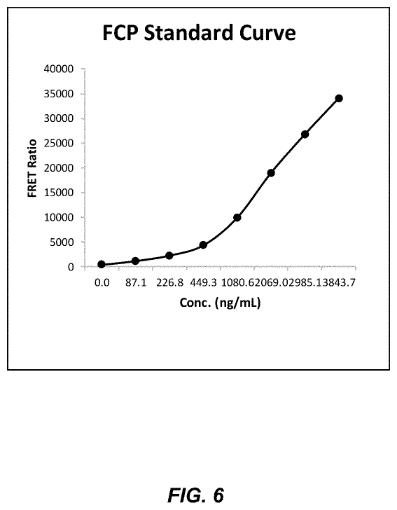

If one anti-calprotectin antibody is labeled with a donor fluorophore and a second anti-calprotectin antibody is labeled with an acceptor fluorophore, TR-FRET can occur in the presence of the calprotectin antigen (analyte) (FIGS. 4A or 4B). The increase in FRET signal of the acceptor is proportional to the level of calprotectin present in the patient's sample (e.g., stool or fecal sample) as interpolated from a known amount of calprotectin calibrators (FIG. 5 or FIG. 6).

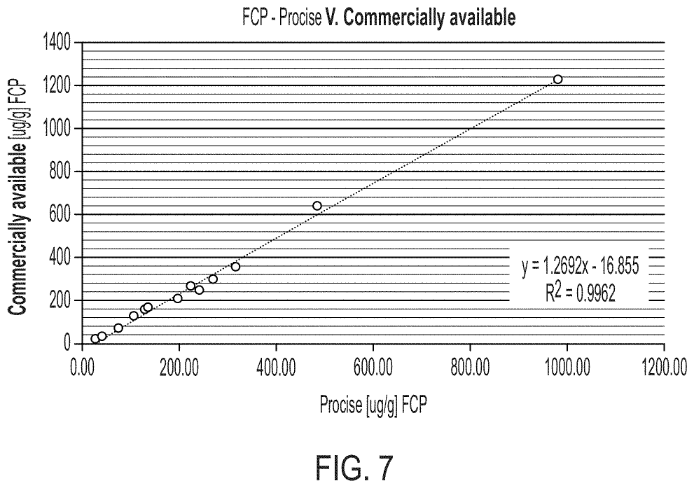

Example 3 illustrates a head to head comparison between the currently it methods (inventive) and a commercially available kit (comparator). The samples were measured in the comparator kit and then compared to the measurements using the inventive method. The measurements using the inventive methods were performed in duplicate. The results are shown in FIG. 7. A R² of 1 indicates that the regression predictions perfectly fit the data. Here, the R² is equal to 0.9962 showing excellent correlation of the inventive methods with comparator.

Example 4 illustrates dye structures and spectral characteristics.

Donor H22TRENIA-5LIO-NHS can be used to label one antibody (FIG. 8). Lumi4 has 4 spectrally distinct peaks, at about 490 nm, about 545 nm, about 580 nm, and about 620 nm, which can be used for energy transfer (FIG. 9). The acceptor molecules that can be used include but are not limited to: AlexaFluor 488, AlexaFluor 546 and AlexaFluor 647 (FIG. 10). Donor and acceptor fluorophores are conjugated using primary amines on antibodies.

Although the foregoing invention has been described in some detail by way of illustration and example for purposes of clarity of understanding, one of skill in the art will appreciate that certain changes and modifications may be practiced within the scope of the appended claims. In addition, each reference provided herein is incorporated by reference in its entirety to the same extent as if each reference was individually incorporated by reference.

INFORMAL SEQUENCE LISTING
>sp|P19320|VCAM1_HUMAN Vascular cell adhesion
protein 1 OS = Homo sapiens OX = 9606 GN = VCAM1
PE = 1 SV = 1
SEQ ID NO: 1:
MPGKMVVILGASNILWIMFAASQAFKIETTPESRYLAQIGDSVSLTCSTTG
CESPFFSWRTQIDSPLNGKVTNEGTTSTLTMNPVSFGNEHSYLCTATCESR
KLEKGIQVEIYSFPKDPEIHLSGPLEAGKPITVKCSVADVYPFDRLEIDLL
KGDHLMKSQEFLEDADRKSLETKSLEVTFTPVIEDIGKVLVCRAKLHIDEM
DSVPTVRQAVKELQVYISPKNTVISVNPSTKLQEGGSVTMTCSSEGLPAPE
IFWSKKLDNGNLQHLSGNATLTLIAMRMEDSGIYVCEGVNLIGKNRKEVEL
IVQEKPFTVEISPGPRIAAQIGDSVMLTCSVMGCESPSFSWRTQIDSPLSG
KVRSEGTNSTLTLSPVSFENEHSYLCTVTCGHKKLEKGIQVELYSFPRDPE
IEMSGGLVNGSSVTVSCKVPSVYPLDRLEIELLKGETILENIEFLEDTDMK
SLENKSLEMTFIPTIEDTGKALVCQAKLHIDDMEFEPKQRQSTQTLYVNVA
PRDTTVLVSPSSILEEGSSVNMTCLSQGFPAPKILWSRQLPNGELQPLSEN
ATLTLISTKMEDSGVYLCEGINQAGRSRKEVELIIQVTPKDIKLTAFPSES
VKEGDTVIISCTCGNVPETWIILKKKAETGDTVLKSIDGAYTIRKAQLKDA
GVYECESKNKVGSQLRSLTLDVQGRENNKDYFSPELLVLYFASSLIIPAIG
MIIYFARKANMKGSYSLVEAQKSKV
SEQ ID NO: 2
MLTELEKALN SIIDVYHKYS LIKGNFHAVY RDDLKKLLET
ECPQYIRKKG ADVWFKELDI NTDGAVNFQE FLILVIKMGV
AAHKKSHEES HKE
SEQ ID NO: 3
MTCKMSQLER NIETIINTFH QYSVKLGHPD TLNQGEFKEL
VRKDLQNFLK KENKNEKVIE HIMEDLDTNA DKQLSFEEFI
MLMARLTWAS HEKMHEGDEG PGHHHKPGLG EGTP

What is claimed is:

1. A method for detecting or quantifying the concentration or level of calprotectin in a sample, the method comprising:
contacting the sample with a first antibody having a first binding epitope specific to calprotectin, wherein the first antibody is labeled with a donor fluorophore;
contacting the sample with a second antibody having a second binding epitope specific to calprotectin, wherein the second antibody is labeled with an acceptor fluorophore;
incubating the sample for a time sufficient to obtain a dual labeled calprotectin; and
exciting the sample having the dual labeled calprotectin using a light source to detect a fluorescent emission signal associated with fluorescence resonance energy transfer (FRET).

2. The method according to claim 1, wherein the FRET emission signal is a time resolved FRET emission signal.

3. The method according to claim 1, wherein the sample is a biological sample.

4. The method according to claim 1, wherein the biological sample is a member selected from the group consisting of whole blood, urine, a fecal specimen, plasma and serum.

5. The method according to claim 1, wherein the biological sample is a fecal specimen.

6. The method according to claim 1, wherein the FRET energy donor compound is a terbium cryptate.

7. The method according to claim 1, wherein the acceptor compound is a member selected from the group consisting of fluorescein-like (green zone) molecule, Cy5, DY-647, Alexa Fluor 488, Alexa Fluor 546, Allophycocyanin (APC), Phycoeruythrin (PE) and Alexa Fluor 647.

8. The method according to claim 7, wherein the acceptor compound is Alexa Fluor 647.

9. The method according to claim 1, wherein the excitation wavelength is between about 300 nm to about 400 nm.

10. The method according to claim 1, wherein the emission wavelength is about 450 nm to 700 nm.

11. The method according to claim 1, wherein the concertation amount of calprotectin is in a range of about 10 μg/g to about 800 μg/g.

12. The method according to claim 11, wherein the concentration amount of calprotectin is in a range of about 10 μg/g to about 100 μg/g.

13. The method according to claim 11, wherein the concentration amount of calprotectin is in a range of about 100 μg/g to about 800 μg/g.

14. A method for determining inflammation of the gut, the method comprising:
contacting the sample with a first antibody having a first binding epitope specific to calprotectin, wherein the first antibody is labeled with a donor fluorophore;
contacting the sample with a second antibody having a second binding epitope specific to calprotectin, wherein the second antibody is labeled with an acceptor fluorophore;
incubating the sample for a time sufficient to obtain a dual labeled calprotectin; and
exciting the sample having the dual labeled calprotectin using a light source to detect a fluorescent emission signal associated with fluorescence resonance energy transfer (FRET).

15. The method of claim 14, wherein the method is used to determine or diagnosis Inflammatory Bowel Disease (IBD).

16. The method of claim 14, wherein the method is used to determine or diagnosis irritable bowel syndrome (IBS).

17. A assay method for detecting the presence or amount of VCAM-1 in a sample, the method comprising:
contacting the sample with a first anti-VCAM-1 antibody having a first binding epitope to VCAM-1, wherein the first anti-VCAM-1 antibody is labeled with a donor fluorophore;
contacting the sample with a second anti-VCAM-1 antibody having a second binding epitope to VCAM-1, wherein the second anti-VCAM-1 antibody is labeled with an acceptor fluorophore;
incubating the sample for a time sufficient to obtain dual labeled VCAM-1; and
exciting the sample having dual labeled VCAM-1 using a light source to detect a fluorescence emission signal associated with fluorescence resonance energy transfer (FRET).

18. The method according to claim 17, wherein the FRET emission signal is a time resolved FRET emission signal.

19. The method according to claim 17, wherein the sample is a biological sample.

20. The method according to claim 17, wherein the biological sample is a member selected from the group consisting of whole blood, urine, a fecal specimen, plasma and serum.


SEQUENCE LISTING (3 sequences)

SEQ ID NO: 1 - protein, 739 aa, Homo sapiens
MPGKMVVILGASNILWIMFAASQAFKIETTPESRYLAQIGDSVSLTCSTTGCESPFFSWRTQIDSPLNGKVTNEGTTSTLTMNPVSFGNEHSYLCTATCESRKLEKGIQVEIYSFPKDPEIHLSGPLEAGKPITVKCSVADVYPFDRLEIDLLKGDHLMKSQEFLEDADRKSLETKSLEVTFTPVIEDIGKVLVCRAKLHIDEMDSVPTVRQAVKELQVYISPKNTVISVNPSTKLQEGGSVTMTCSSEGLPAPEIFWSKKLDNGNLQHLSGNATLTLIAMRMEDSGIYVCEGVNLIGKNRKEVELIVQEKPFTVEISPGPRIAAQIGDSVMLTCSVMGCESPSFSWRTQIDSPLSGKVRSEGTNSTLTLSPVSFENEHSYLCTVTCGHKKLEKGIQVELYSFPRDPEIEMSGGLVNGSSVTVSCKVPSVYPLDRLEIELLKGETILENIEFLEDTDMKSLENKSLEMTFIPTIEDTGKALVCQAKLHIDDMEFEPKQRQSTQTLYVNVAPRDTTVLVSPSSILEEGSSVNMTCLSQGFPAPKILWSRQLPNGELQPLSENATLTLISTKMEDSGVYLCEGINQAGRSRKEVELIIQVTPKDIKLTAFPSESVKEGDTVIISCTCGNVPETWIILKKKAETGDTVLKSIDGAYTIRKAQLKDAGVYECESKNKVGSQLRSLTLDVQGRENNKDYFSPELLVLYFASSLIIPAIGMIIYFARKANMKGSYSLVEAQKSKV

SEQ ID NO: 2 - protein, 93 aa, Homo sapiens
MLTELEKALNSIIDVYHKYSLIKGNFHAVYRDDLKKLLETECPQYIRKKGADVWFKELDINTDGAVNFQEFLILVIKMGVAAHKKSHEESHKE

SEQ ID NO: 3 - protein, 114 aa, Homo sapiens
MTCKMSQLERNIETIINTFHQYSVKLGHPDTLNQGEFKELVRKDLQNFLKKENKNEKVIEHIMEDLDTNADKQLSFEEFIMLMARLTWASHEKMHEGDEGPGHHHKPGLGEGTP